(19) United States
(12) Patent Application Publication
(10) Pub. No.: US 2022/0020464 A1
(43) Pub. Date: Jan. 20, 2022

(54) SYSTEM AND METHOD FOR PREDICTIVE DETERMINATION OF FACTORS THAT AFFLICT MENTAL STATE OF A USER

(71) Applicants: Aleksander ZIVKOVIC, Toronto (CA); Marvin IGELMAN, Thornhill (CA)

(72) Inventors: Aleksander ZIVKOVIC, Toronto (CA); Marvin IGELMAN, Thornhill (CA)

(21) Appl. No.: 17/377,027

(22) Filed: Jul. 15, 2021

Related U.S. Application Data
(60) Provisional application No. 63/052,568, filed on Jul. 16, 2020.

(51) Int. Cl. G16H 20/10; G16H 10/60; G16H 50/20; G16H 50/30; G06N 20/00; G06K 9/62

(52) U.S. Cl. CPC G16H 20/10; G16H 10/60; G06K 9/6256; G16H 50/30; G06N 20/00; G16H 50/20

(57) ABSTRACT

There is provided a method and system for predictive determination of factors that afflict mental state of a user. The method includes receiving data associated with information that can afflict a mental state of the user; receiving data associated with events affecting the user; determining anomalies in a mental state of the user using a trained first machine learning model, the first machine learning model outputting a binary classifier indicating either that the user is in a normal mental state and or that the user is in an anomalous mental state; and determining a mental state of the user using a trained second machine learning model, the second machine learning model outputting a probability of the mental state of the user being either ordinary or extraordinary.

### TECHNICAL FIELD

The following relates generally to predictions of biometric information and more specifically to a system and method for predictive determination of factors that afflict mental state of a user.

### BACKGROUND

Nutraceuticals are compounds that can promote mental health and that can be taken in safety; however, one should not abuse such compounds and should use them sporadically and at an appropriate time. Nutraceuticals have the ability to alter mood, and generally impact the mental state of an individual, and a significant challenge is taking them safely and effectively.

### SUMMARY

In an aspect, there is provided a computer-implemented method for predictive determination of factors that afflict mental state of a user, the method comprising: receiving data associated with information that can afflict a mental state of the user; receiving data associated with events affecting the user; determining anomalies in a mental state of the user using a trained first machine learning model, the first machine learning model trained using at least a subset of the data associated with information that can afflict a mental state of the user and the data associated with events affecting the user, the first machine learning model outputting a binary classifier indicating either that the user is in a normal mental state and or that the user is in an anomalous mental state; determining a mental state of the user using a trained second machine learning model, the second machine learning model trained using the determined anomalies and at least a subset of the data associated with information that can afflict a mental state of the user and the data associated with events affecting the user, the subset of the data associated with information that can afflict a mental state of the user comprises user input with respect to the mental state of the user, the second machine learning model outputting a probability of the mental state of the user being either ordinary or extraordinary; and outputting at least one of the binary classifier and the probability of the mental state of the user.

In a particular case of the method, the first machine learning model comprises one of an AutoRegressive Integrated Moving Average (ARIMA), a long short-term memory (LSTM), or a Hidden Markov Models (HMM).

In another case of the method, the second machine learning model comprises one of a feed-forward neural net, a long short-term memory (LSTM), a gated recurrent unit (GRU), a convolutional neural network (CNN)).

In yet another case of the method, the method further comprising: determining an effect of a nutraceutical on the mental state of the user using a trained third machine learning model, the third machine learning model trained using the determined mental state of the user and at least a subset of the data associated with information that can afflict a mental state of the user and the data associated with events affecting the user, the subset of the data associated with information that can afflict a mental state of the user comprises user input with respect to consumption of the nutraceutical, the third machine learning model outputting a future path prediction of the mental state of the user for a given current mental state in a consumption of a nutraceutical; and outputting the future path prediction of the mental state of the user.

In yet another case of the method, the user input with respect to consumption of the nutraceutical comprises a schedule of when the nutraceutical was consumed and an associated dosage, the mental state of the user prior to consumption, and the mental state of the user after the consumption.

In yet another case of the method, the third machine learning model comprises a regression model.

In yet another case of the method, the method further comprising: determining a desired dosage and a time of application of the nutraceutical to minimize negative effect on the mental state of the user using a trained fourth machine learning model, the fourth machine learning model trained using the determined future path prediction of the mental state of the user and at least a subset of the data associated with information that can afflict a mental state of the user and the data associated with events affecting the user, the third machine learning model outputting a set of control signals for dosage and a time of application of the nutraceutical by the user to avoid an undesired mental state change; and outputting the set of control signals.

In yet another case of the method, a cost function for the fourth machine learning model is based on a difference between a desired mental state and an observed mental state.

In yet another case of the method, the fourth machine learning model comprises drop-out layers for neural networks and uses train-test-validation separation by either using stratified-k-folds or purged-k-folds.

In yet another case of the method, the control signals further comprise suggestions to the user to perform a mental stimulating activity.

In another aspect, there is provided a system for predictive determination of factors that afflict mental state of a user, the system in communication with a measurement device, the system comprising one or more processors and a memory, the one or more processors configured to execute: a collection module to receive data associated with information that can afflict a mental state of the user from the measurement device and data associated with events affecting the user; a first machine learning module to determine anomalies in a mental state of the user using a trained first machine learning model, the first machine learning model trained using at least a subset of the data associated with information that can afflict a mental state of the user and the data associated with events affecting the user, the first machine learning model outputting a binary classifier indicating either that the user is in a normal mental state and or that the user is in an anomalous mental state; a second machine learning module to determine a mental state of the user using a trained second machine learning model, the second machine learning model trained using the determined anomalies and at least a subset of the data associated with information that can afflict a mental state of the user and the data associated with events affecting the user, the subset of the data associated with information that can afflict a mental state of the user comprises user input with respect to the mental state of the user, the second machine learning model outputting a probability of the mental state of the user being either ordinary or extraordinary; and an output module to output at least one of the binary classifier and the probability of the mental state of the user.

In a particular case of the system, the first machine learning model comprises one of an AutoRegressive Integrated Moving Average (ARIMA), a long short-term memory (LSTM), or a Hidden Markov Models (HMM).

In another case of the system, the second machine learning model comprises one of a feed-forward neural net, a long short-term memory (LSTM), a gated recurrent unit (GRU), a convolutional neural network (CNN)).

In yet another case of the system, the system further comprising: a third machine model to determine an effect of a nutraceutical on the mental state of the user using a trained third machine learning model, the third machine learning model trained using the determined mental state of the user and at least a subset of the data associated with information that can afflict a mental state of the user and the data associated with events affecting the user, the subset of the data associated with information that can afflict a mental state of the user comprises user input with respect to consumption of the nutraceutical, the third machine learning model outputting a future path prediction of the mental state of the user for a given current mental state in a consumption of a nutraceutical, wherein the output module further outputs the future path prediction of the mental state of the user.

In yet another case of the system, the user input with respect to consumption of the nutraceutical comprises a schedule of when the nutraceutical was consumed and an associated dosage, the mental state of the user prior to consumption, and the mental state of the user after the consumption.

In yet another case of the system, the third machine learning model comprises a regression model.

In yet another case of the system, the system further comprising: a fourth machine learning model to determine a desired dosage and a time of application of the nutraceutical to minimize negative effect on the mental state of the user using a trained fourth machine learning model, the fourth machine learning model trained using the determined future path prediction of the mental state of the user and at least a subset of the data associated with information that can afflict a mental state of the user and the data associated with events affecting the user, the third machine learning model outputting a set of control signals for dosage and a time of application of the nutraceutical by the user to avoid an undesired mental state change, wherein the output module further outputs the set of control signals.

In yet another case of the system, a cost function for the fourth machine learning model is based on a difference between a desired mental state and an observed mental state.

In yet another case of the system, the fourth machine learning model comprises drop-out layers for neural networks and uses train-test-validation separation by either using stratified-k-folds or purged-k-folds.

In yet another case of the system, the control signals further comprise suggestions to the user to perform a mental stimulating activity.

These and other aspects are contemplated and described herein. It will be appreciated that the foregoing summary sets out representative aspects of the embodiments and assists skilled readers in understanding the following detailed description.

### DESCRIPTION OF THE DRAWINGS

A greater understanding of the embodiments will be had with reference to the Figures, in which:

FIG. 1 is a schematic diagram of a system for predictive determination of factors that afflict mental state, in accordance with an embodiment;

FIG. 2 is a schematic diagram showing the system of FIG. 1 and an exemplary operating environment;

FIG. 3 is a flow chart of a method for predictive determination of factors that afflict mental state, in accordance with an embodiment; and

FIG. 4 is a diagrammatic example of predictive model control.

### DETAILED DESCRIPTION

Embodiments will now be described with reference to the figures. For simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

Any module, unit, component, server, computer, terminal or device exemplified herein that executes instructions may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the device or accessible or connectable thereto. Any application or module herein described may be implemented using computer readable/executable instructions that may be stored or otherwise held by such computer readable media.

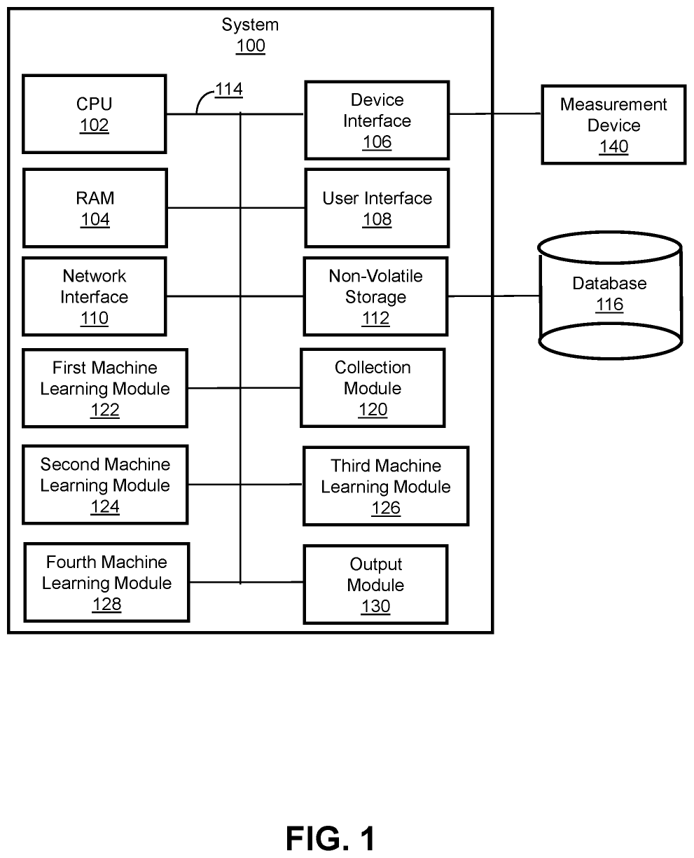

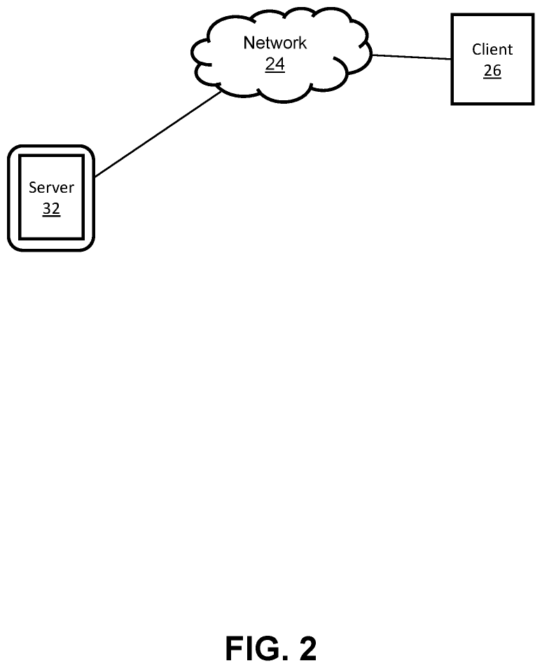

Referring now to FIG. 1 and FIG. 2, a system 100 for predictive determination of factors that afflict mental state, in accordance with an embodiment, is shown. In this embodiment, the system 100 is run on a client side device 26 and accesses content located on a server 32 over a network 24, such as the internet. In further embodiments, the system 100 can be run on any suitable computing device; for example, a desktop computer, a laptop computer, a smartphone, a tablet computer, the server 32, distributed or cloud computing device(s), or the like. In some embodiments, the components of the system 100 are stored by and executed on a single computer system. In other embodiments, the components of the system 100 are distributed among two or more computer systems that may be locally or remotely distributed.

FIG. 1 shows various physical and logical components of an embodiment of the system 100. As shown, the system 100 has a number of physical and logical components, including a central processing unit (“CPU”) 102 (comprising one or more processors), random access memory (“RAM”) 104, a device interface 106, a user interface 108, a network interface 110, non-volatile storage 112, and a local bus 114 enabling CPU 102 to communicate with the other components. CPU 102 executes an operating system, and various conceptual modules, as described below in greater detail. RAM 104 provides relatively responsive volatile storage to CPU 102. The user interface 108 enables an administrator or user to provide input via an input device, for example a keyboard and mouse. In other cases, the input data can be already located on the database 116 or received via the network interface 110. The user interface 108 can also output information to output devices, for example, a display. The device interface 106 permits the system 100 to communicate with measurement devices 140, such as wearable devices described herein. The network interface 110 permits communication with other systems, such as other computing devices and servers remotely located from the system 100, such as for a typical cloud-based access model. Non-volatile storage 112 stores the operating system and programs, including computer-executable instructions for implementing the operating system and modules, as well as any data used by these services. Additional stored data can be stored in a database 116. During operation of the system 100, the operating system, the modules, and the related data may be retrieved from the non-volatile storage 112 and placed in RAM 104 to facilitate execution.

In an embodiment, the CPU 102 is configurable to execute a number of functional modules, such as a collection module 120, a first machine learning module 122, a second machine learning module 124, a third machine learning module 126, a fourth machine learning module 128, and an output module 130. In further cases, the functions of any of the modules can be combined, further separated, or part of different modules.

In a particular case, a user has access to a measurement device 140; such as a wearable device, for example, a watch, phone, or specific device that interacts with user and obtains sensory information. Such sensory information can include some of following factors but is not limited to, their sleep patterns, pedometer, heart rate monitor, blood pressure and other information. It is also assumed that the user has access to external factors that might affect their mental state, such as news about world events, weather at their locality, local events, and the like.

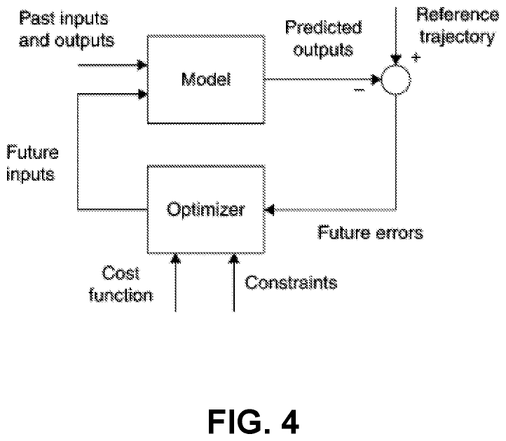

The system 100 is used to share users own biometric signals as well as communicate with the user when an anomaly is detected in their biometric signals in order to determine the potential cause of the anomaly. This can be done through the user interface 108 via, for example, a chat bot, or through voice interaction, where the user is asked about what particular factor is contributing to their distress; whether it is personal, some global event, or some local environmental factor. By utilizing the aforementioned information as in input to a machine learning model, the system 100 can have predictive capabilities, that can predict users' likely behavior in the presence of future events, either global or local that could affect their mental state of being. Similarly, it is possible to track the change in users change in mental state when they have taken nutraceuticals, and also obtain this information. Tracking of the change in the biometric information, and correlating it with the confirmed nutraceutical consumption (for example, as inputted into the user interface 108) provides a dataset that can be used for training the machine learning model that can predict effect of a nutraceutical (including dosage) on the mental state of an individual. By combining the predictions for the change in behavior from the user when exposed to certain external elements, and by assuming that the user will be exposed to such external factors (global news, local news, weather, etc.) with the prediction of the effect of the nutraceuticals on the individual, a predictive model control can be implemented where the nutraceutical (such as neurocetical) dosage is suggested in advance of the user of actual learning the event, thereby reducing significant anomalies in users mental state. This can be implemented as a specific type of predictive model control, as diagrammatically exemplified in FIG. 4, where the user's mental state is modeled based on past inputs and outputs, a reference trajectory is devoid of sudden change in mental state reflected through rapid change in biometrics, and an optimizer identifies most appropriate dosages of the control (neurocetical) substance that predictively impacts the user ahead of the exposure to distressing events.

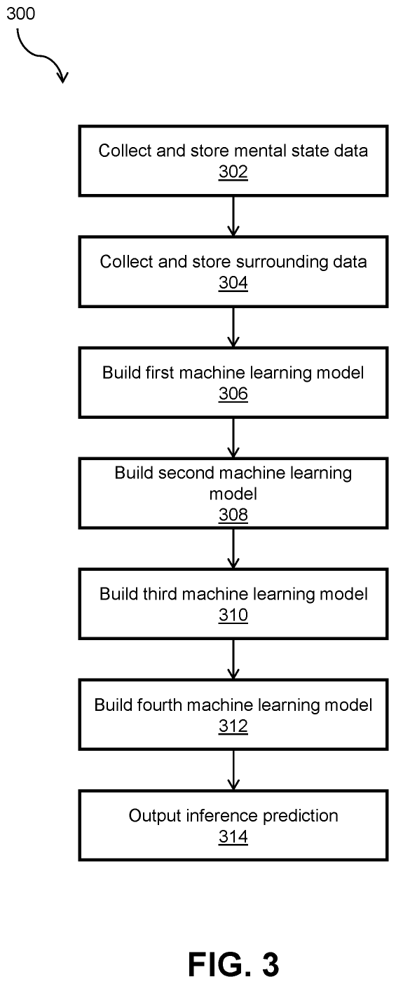

FIG. 3 illustrates a flowchart of a method for predictive determination of factors that afflict mental state 300, in accordance with an embodiment.

At block 302, the collection module 120 collects and stores data associated with information that can afflict a user's mental state. The measurement signals can be collected and either stored on the local device (such as in the database 116) or directly transferred in real-time to servers via the network interface 110 through secured channels that are tasked with collecting all of the users real-time signals

The data that is collected about the user can include at least one of (but is not limited to):
Biometric data, that is obtained directly from measurement devices 140 that are either wearable or peripheral devices that can communicate with the users mobile device. Some of these devices are always on, meaning that they continuously obtain information about the user such as heartbeat, temperature, blood pressure while others are obtained on demands such as blood sugar levels or other blood test related results. In some cases the wearables might be devices such as implants, or neurosynaptic connection devices, or external electromagnetic devices that can tap into electromagnetic field of the brain which can further be interpreted through pattern detection to interpret brain activity. In other cases the device might be a smart contact lens that obtains readings from the user's eye or smart glasses that are interpreting the user's eye motion.
Other data from a mobile device measurement device 140: for example, sleep analysis, pedometer measurement, exercise routines, calendar information, as well as users location which can be used for determining local weather information such as barometric pressure, temperature, humidity etc.
Communication data, which is data that is obtained through the communication with the user via the user interface 108. The communication can be done through a chat bot to try to obtain users mental state information, or it could be done through voice communication, where the user communicates through a natural language conversation. The communication logic can be either implemented directly on the users mobile device or it could be implemented through server logic that communicates with the user mobile device. All of the communication is either stored on the device for later transmission, or transferred in real-time to the servers through secured connection for immediate access.
Users health records: these are either health records that the user shares directly via the user interface 108 or allow access to medical records within a networked health system via the network interface 110.
Users scans: these are MRI, CT and other scans of the brains activity that the user has or grants access to via the user interface 108.
Lab results: any results of tests that are done in the lab and inputted via the user interface 108 or via the network interface 110.
Medication and medication schedule: Any medications that the user is taking and schedule as to when the medication is taken, and inputted via the user interface 108 or via the network interface 110.
Nutraceuticals schedule: when and what nutraceuticals have been consumed by the user as inputted via the user interface 108.

At block 304, the collection module 120 collects and stores data associated with the world surrounding the user, such as data inputted via the user interface 108 and/or received over the network interface 110. Such surrounding data can include at least one of (but is not limited to):
Significant world events such as political news, terrorism, natural disasters, sports results, news in entertainment and music, and other general news, that is obtained in real-time from either social media or news channels, as a stream of events that might potentially affect users state of mind. Source of this type of information could be a published event stream from companies such as Evntl™, DataMinr™, and the like, combined with RSS feeds from news organizations.
Local news events obtained in real-time from local news sources, social media, and other online sources, harvested in real-time and correlated with a user's location in order to determine what might be happening in close proximity to the user. Such information could be a published event stream for location information from companies such as Evntl™, DataMinr™, local news organizations RSS, and information from local fire and police Twitter™ accounts.
Local weather conditions with respect to the user's location.
Traffic information around the user's location.
Natural conditions with respect to user's location such as lunar phase, water levels, pollution index, and the like.

At block 306, the first machine learning module 122 uses the data collected about the user and the surrounding data to build a first machine learning model. The first machine learning model is used to detect anomalies in the user's mental state. In some cases, time series model techniques are utilized to monitor the user's biometrics which have an established pattern when unaffected by special conditions. Models that are particularly well suited for detecting anomalies in time series data include, for example, AutoRegressive Integrated Moving Average (ARIMA) and long short-term memory (LSTM). Hidden Markov Models (HMMs) can also be used to determine normal versus abnormal regimes in data. Similarly, matrix profiles can be used to detect abnormalities in patterns. Once the anomalies are detected, they are marked in the sequence and corresponding state of the data prior to the anomaly are located to represent the data state with all the features prior to the occurrence of the anomaly. The output of this model is the probability prediction of whether a certain point or set of points in the time series is an anomaly. The model is therefore a binary classifier with 0 being normal state and 1 being anomaly. Note that this model is utilized as a way of generating training data for the user's mental state prediction model.

At block 308, the second machine learning module 124 builds a second machine learning model that predicts users mental state based on the data collected about the user and the surrounding data. In order to create this type of model, the information from all of the data channels are correlated with the user input with respect to their mental state. All the non-numeric data is converted to numeric vectors using embedding; for natural language data such as news events, the embedding can be done using simple word embedding mechanism such as word2vec, glove, or tf-idf with bag-of-words mechanism; however, more complex embeddings which represent each sentence as a vector are preferable, such as output of hidden layers of complex models trained on very large corpora (for example, universal sentence embedding, BERT and other transfer learning models). The anomalies detected by the first machine learning model at block 306, and the associated data preceding the anomaly, are used by the second machine learning module 124 to build a more complex, non-linear model that can handle a large number of features. The second machine learning model can be a feed-forward neural net (multi-layer perceptron), deep learning models with various architectures (LSTM, gated recurrent unit (GRU), convolutional neural network (CNN)) that include drop-out layers in order to minimize overfitting as well as simper models such as decision trees, gradient boosted trees, random forests and XGBOOST. The k-fold sampling is used to generate out-of-sample predictions from various models and second level model (typically a neural net or a random forest) is utilized to combine the predictions from the first layer models to create a final prediction. The second machine learning model is utilized to predict possibility of mental state changed based on the available inputs obtainable in real-time from user and public information. The output of this model is the probability of the mental state being 0 or 1 (binary classifier model output) with 0 being ordinary and 1 being extraordinary.

At block 310, the third machine learning module 126 builds a third machine learning model to predict the effect of nutraceuticals on the user's mental state. The third machine learning model is a model that is trained based on information regarding effect of the nutraceuticals on the user based on the data collected about the user and the surrounding data. By obtaining the schedule of when the nutraceutical was consumed, with dosage, the state of the user prior, and the state of the user after the consumption, with all the associated biometric of the user, the third machine learning model can be trained to show the predicted effect of the nutraceutical and time that the effect will likely take. The third machine learning model can be based on assumptions in terms of distribution of the active ingredient within the body. Both aggregate information from all of the patients consuming the nutraceutical for which the data is available can be used to generate an overall model of the effect on the population in general, as well as with enough samples from a user, a user specific model can be created. The input to the third machine learning model is therefore dosage and time of when a nutraceutical was taken, or time of when a mental exercise was done, and the output is the effect on user mental state as measured through biometrical signals. Note that it is possible that a further machine learning model may be required for interpreting the biometric signals in terms of mental state, however, in most cases, it can be assumed that there is a strong correlation between biometric signals and mental state. The output of the third machine learning model is the effect on the mental state in terms of response, so the third machine learning model is a regression model that makes future path prediction of mental state for given current state and possible therapeutic nutraceutical and/or mental exercise or activity.

At block 312, the fourth machine learning module 128 trains a fourth machine learning model for optimization given the current state of all the user parameters, the knowledge of the events and conditions around the user, the prediction of how the user will likely respond to the environmental changes, and the output of the third machine learning model that predicts the users likely state changes when taking nutraceuticals. The fourth machine learning model predicts a desired dosage and a time of application to minimize negative effect on the user. The cost function for the fourth machine learning model is based on difference between desired mental state as reflected by biometric signal and observed mental state. The output of the fourth machine learning model is the actual set of control signals that are suggested to the end user to apply in real-time, which are meant to avoid the undesired mental state changes for the user in the future.

In some cases, the above machine learning models can be continuously updated as more input information becomes available.

At block 314, during an inference stage, the output module 130 outputs the determined predictions from one or more of the machine learning models to the user interface 108, to the database 116, to the device interface 106, or to the network interface 110. In some cases, both models made from aggregate data are created as well as user specific models, and the output of the two types of models can be combined when performing inference.

With the pre-trained models available, the data can be collected continuously, predictions can be made as the information becomes available and the fourth machine learning model can suggest control signals that can prevent undesired changes in the mental state. Most common models include Kalman filters but more complex models can be utilized as well as long as care is taken not to overfit the available data (which is achieved through adding drop-out layers for neural nets and use of proper train-test-validation separation in the data by either using stratified-k-folds or in case of time series analysis purged-k-folds where an embargo area is introduced in the time slice where possible overlap between the signal and ground truth is possible).

The control signals can be anything that affects the mental state of the user. This could be suggestion of doing mental exercises such as meditation, some type of mental activity that is calming to the user and focuses their attention such as game of chess, or other mentally stimulating game, calming music, or videos that have been previously identified during the training process to help with mental state of the user. The control signal could also suggest taking nutraceuticals and or other chemical-based compounds that are approved for human usage, be it prescription drugs or nutraceuticals, that are scheduled with specific dosage and lead time to perhaps obtaining knowledge of the event that might be upsetting to the user. The control signal might include voluntary or involuntary denial of access to the mobile device and/or other devices that might reveal potentially destabilizing event to the user ahead of desired time for the effect of the other activity/nutraceutical to take place.

Although the invention has been described with reference to certain specific embodiments, various modifications thereof will be apparent to those skilled in the art without departing from the spirit and scope of the invention as outlined in the claims appended hereto.

1. A computer-implemented method for predictive determination of factors that afflict mental state of a user, the method comprising:
receiving data associated with information that can afflict a mental state of the user;
receiving data associated with events affecting the user;
determining anomalies in a mental state of the user using a trained first machine learning model, the first machine learning model trained using at least a subset of the data associated with information that can afflict a mental state of the user and the data associated with events affecting the user, the first machine learning model outputting a binary classifier indicating either that the user is in a normal mental state and or that the user is in an anomalous mental state;
determining a mental state of the user using a trained second machine learning model, the second machine learning model trained using the determined anomalies and at least a subset of the data associated with information that can afflict a mental state of the user and the data associated with events affecting the user, the subset of the data associated with information that can afflict a mental state of the user comprises user input with respect to the mental state of the user, the second machine learning model outputting a probability of the mental state of the user being either ordinary or extraordinary; and
outputting at least one of the binary classifier and the probability of the mental state of the user.

2. The method of claim 1, wherein the first machine learning model comprises one of an AutoRegressive Integrated Moving Average (ARIMA), a long short-term memory (LSTM), or a Hidden Markov Models (HMM).

3. The method of claim 1, wherein the second machine learning model comprises one of a feed-forward neural net, a long short-term memory (LSTM), a gated recurrent unit (GRU), a convolutional neural network (CNN)).

4. The method of claim 1, further comprising:
determining an effect of a nutraceutical on the mental state of the user using a trained third machine learning model, the third machine learning model trained using the determined mental state of the user and at least a subset of the data associated with information that can afflict a mental state of the user and the data associated with events affecting the user, the subset of the data associated with information that can afflict a mental state of the user comprises user input with respect to consumption of the nutraceutical, the third machine learning model outputting a future path prediction of the mental state of the user for a given current mental state in a consumption of a nutraceutical; and
outputting the future path prediction of the mental state of the user.

5. The method of claim 4, wherein the user input with respect to consumption of the nutraceutical comprises a schedule of when the nutraceutical was consumed and an associated dosage, the mental state of the user prior to consumption, and the mental state of the user after the consumption.

6. The method of claim 4, wherein the third machine learning model comprises a regression model.

7. The method of claim 4, further comprising:
determining a desired dosage and a time of application of the nutraceutical to minimize negative effect on the mental state of the user using a trained fourth machine learning model, the fourth machine learning model trained using the determined future path prediction of the mental state of the user and at least a subset of the data associated with information that can afflict a mental state of the user and the data associated with events affecting the user, the third machine learning model outputting a set of control signals for dosage and a time of application of the nutraceutical by the user to avoid an undesired mental state change; and
outputting the set of control signals.

8. The method of claim 7, wherein a cost function for the fourth machine learning model is based on a difference between a desired mental state and an observed mental state.

9. The method of claim 7, wherein the fourth machine learning model comprises drop-out layers for neural networks and uses train-test-validation separation by either using stratified-k-folds or purged-k-folds.

10. The method of claim 7, wherein the control signals further comprise suggestions to the user to perform a mental stimulating activity.

11. A system for predictive determination of factors that afflict mental state of a user, the system in communication with a measurement device, the system comprising one or more processors and a memory, the one or more processors configured to execute:
a collection module to receive data associated with information that can afflict a mental state of the user from the measurement device and data associated with events affecting the user;
a first machine learning module to determine anomalies in a mental state of the user using a trained first machine learning model, the first machine learning model trained using at least a subset of the data associated with information that can afflict a mental state of the user and the data associated with events affecting the user, the first machine learning model outputting a binary classifier indicating either that the user is in a normal mental state and or that the user is in an anomalous mental state;
a second machine learning module to determine a mental state of the user using a trained second machine learning model, the second machine learning model trained using the determined anomalies and at least a subset of the data associated with information that can afflict a mental state of the user and the data associated with events affecting the user, the subset of the data associated with information that can afflict a mental state of the user comprises user input with respect to the mental state of the user, the second machine learning model outputting a probability of the mental state of the user being either ordinary or extraordinary; and
an output module to output at least one of the binary classifier and the probability of the mental state of the user.

12. The system of claim 11, wherein the first machine learning model comprises one of an AutoRegressive Integrated Moving Average (ARIMA), a long short-term memory (LSTM), or a Hidden Markov Models (HMM).

13. The system of claim 11, wherein the second machine learning model comprises one of a feed-forward neural net, a long short-term memory (LSTM), a gated recurrent unit (GRU), a convolutional neural network (CNN)).

14. The system of claim 11, further comprising:
a third machine model to determine an effect of a nutraceutical on the mental state of the user using a trained third machine learning model, the third machine learning model trained using the determined mental state of the user and at least a subset of the data associated with information that can afflict a mental state of the user and the data associated with events affecting the user, the subset of the data associated with information that can afflict a mental state of the user comprises user input with respect to consumption of the nutraceutical, the third machine learning model outputting a future path prediction of the mental state of the user for a given current mental state in a consumption of a nutraceutical, wherein the output module further outputs the future path prediction of the mental state of the user.

15. The system of claim 14, wherein the user input with respect to consumption of the nutraceutical comprises a schedule of when the nutraceutical was consumed and an associated dosage, the mental state of the user prior to consumption, and the mental state of the user after the consumption.

16. The system of claim 14, wherein the third machine learning model comprises a regression model.

17. The system of claim 14, further comprising:
a fourth machine learning model to determine a desired dosage and a time of application of the nutraceutical to minimize negative effect on the mental state of the user using a trained fourth machine learning model, the fourth machine learning model trained using the determined future path prediction of the mental state of the user and at least a subset of the data associated with information that can afflict a mental state of the user and the data associated with events affecting the user, the third machine learning model outputting a set of control signals for dosage and a time of application of the nutraceutical by the user to avoid an undesired mental state change, wherein the output module further outputs the set of control signals.

18. The system of claim 17, wherein a cost function for the fourth machine learning model is based on a difference between a desired mental state and an observed mental state.

19. The system of claim 17, wherein the fourth machine learning model comprises drop-out layers for neural networks and uses train-test-validation separation by either using stratified-k-folds or purged-k-folds.

20. The system of claim 17, wherein the control signals further comprise suggestions to the user to perform a mental stimulating activity.
